(19) United States
(12) Patent Application Publication
(10) Pub. No.: US 2015/0258055 A1
(43) Pub. Date: Sep. 17, 2015

(54) METHODS FOR MODULATING CORTISOL LEVELS USING GREEN COFFEE BEAN EXTRACT

(71) Applicant: Applied Food Sciences, Inc., Austin, TX (US)

(72) Inventor: Christine C. Fields, Janesville, WI (US)

(21) Appl. No.: 14/658,879

(22) Filed: Mar. 16, 2015

Related U.S. Application Data
(60) Provisional application No. 61/953,815, filed on Mar. 15, 2014.

(51) Int. Cl. A61K 31/235; A61K 36/74

(52) U.S. Cl. CPC A61K 31/235; A61K 36/74

(57) ABSTRACT

Methods to modulate/reduce cortisol levels in humans are described. Compositions comprising therapeutically effective amount green coffee bean extract, the green coffee extract containing at least 20% chlorogenic acids by dry weight, are administered to human subjects to reduce levels.

### STATEMENT OF PRIORITY

The present application claims priority to U.S. Provisional Application No. 61/953,815, titled “Methods For Modulating Cortisol Levels Using Green Coffee Bean Extract” and filed Mar. 15, 2014.

### FIELD OF THE INVENTION

The present disclosure relates to methods to modulate (reduce) naturally recirculating levels of cortisol in human subjects using green coffee bean extract.

### BACKGROUND OF THE INVENTION

Cortisol, a glucocorticoid hormone secreted by the adrenal cortex, is responsible for the regulation of fat, carbohydrate, and protein metabolism. In humans, cortisol is the main circulating glucocorticoid and it is involved in different physiological functions such as sleep cycle regulation, metabolism, immunity, mood normalization, memorization and learning. Variations in plasma cortisol concentration are also associated with diseases of the musculoskeletal, gastrointestinal, cardiovascular, endocrine and central nervous systems.

Adrenal fatigue is prevalent in today's society, though it was first described in textbooks over a century ago. The term refers to exhaustion of adrenal glands, which secrete two hormones related to stress level: Cortisol and DHEA. Affected individuals have elevated Cortisol levels and depressed DHEA levels, and may suffer from a broad spectrum of non-specific yet debilitating symptoms, such as low energy, sleep problems, weight gain, memory loss, and susceptibility to infections. For example, commercially available extracts (e.g. Relora®) although capable of improving Cortisol and DHEA levels in the body are limited in use due to the side effects. Garrison et al., Alternative Therapies in Heath and Medicine 2006, 12(1):50-54. Hence, there is a need for compositions which improve the delivery and/or reduce the side effects of these medicinal extracts.

Excessive cortisol synthesis leads to changes in metabolism, cognitive impairment (McEwen, 1994) and immunosuppression (Chrousos and Gold, 1992). Abnormalities at different levels of the hypothalamic-pituitary-adrenal (HPA) axis have been reported in several diseases, such as psychiatric disorders, including depression and mood alteration (Kiraly et al., 1997; Tafet et al., 2001), acquired immunodeficiency syndrome (AIDS) (Corley, 1996; Bhansali et al., 2000; Christeff et al., 2000), multiple sclerosis (Erkut et al, 2002), dementia (Maeda et al., 1991; Polled et al., 2002), Alzheimer's disease (Swaab et al., 1994; O'Brien et al., 1996; Weiner et al., 1997; Giubilei et al., 2001; Rasmuson et al., 2002), and breast cancer outcome (Luecken et al, 2002). Disruption of hormonal balance in these diseases leads to increased cortisol production resulting in elevated concentrations of cortisol in cerebrospinal fluid (Swaab et al., 1994; Erkut et al, 2002), blood (Weiner et al., 1997; Bhansali et al., 2000; Rasmuson et al., 2002), urine (Maeda et al., 1991) and saliva (Giubilei et al., 2001).

At the cellular level, glucocorticoids such as cortisol have been shown to decrease cytochrome c oxidase activity (Simon et ah, 1998) and to induce apoptosis in various cell lines (Montague and Cidlowski, 1995). Cortisol is derived from cholesterol. Steroidogenesis begins with the mobilization of free cholesterol and transport from intracellular stores into mitochondria where cholesterol will be metabolized into pregnenolone by the first enzyme of the pathway, the cytochrome P-450 side-chain cleavage enzyme complex (P-450scc). Hormones, such as corticotrophin (ACTH) and its second messenger cAMP (adenosine 3′,5′-cyclic phosphate), acting through the cAMP-dependent protein kinase (PKA), accelerate this process. Although cholesterol transport into mitochondria is the rate-determining step in steroid biosynthesis, steroid formation is also limited by the amount of the substrate cholesterol available.

Cholesterol availability depends on the rate of its synthesis and thus, the activity of the rate-limiting enzyme 3-hydroxy-3-methylglutaryl-coenzyme A (HMG-CoA) reductase located in the cytoplasm, responsible for the conversion of HMG-CoA to mevalonate. Hypercholesterolemia is one of the main reasons for various pathologies of the cardiovascular system. At present, statins are used as the major therapeutic means for hypercholesterolemia because they occupy a portion of the binding site of HMG-CoA, thus blocking access of this substrate to the active site of HMG-CoA reductase (Istvan and Deisenhofer, 2001). In addition, statins are in clinical trials for their use to slow Alzheimer's disease progression, a disease where hypercholesterolemia seems to play a critical role (Waldman and Kitharides, 2003). However, it has been recently reported that statins have numerous serious side effects. Other proposed therapies focus on blocking the activity of enzymes involved in the steroidogenic pathway. However, such methods and compositions result in the inhibition of physiological basal steroid synthesis and may cause detrimental effects to the health of the subject.

HIV-associated dementia (also known as HIV-Associated Dementia Complex, HIV-associated cognitive/motor complex, and AIDS Dementia Complex) is a progressive neurological disorder that affects approximately 58,000 individuals infected with the Human Immunodeficiency Virus (HIV) in the United States. HIV-associated dementia is thought to be a subcortical dementia characterized by cognitive, motor and behavioral impairments severe enough to interfere with an individual's ability to function occupationally or socially.

Early manifestations of HIV-associated dementia may be characterized by cognitive impairment, loss of motor skills, and/or behavioral challenges: Cognitive Impairment, memory loss, impaired concentration, and mental slowing characterized by such actions as slow response are common attributes associated with cognitive impairment. Loss of Motor Skills: Individuals experiencing difficulty with their balance, lack of coordination, leg weakness, clumsiness, poor gait, and/or deteriorating handwriting may be showing signs of deteriorating motor skills. Behavioral Challenges: Uncharacteristic behavior, poor decision-making, personality and mood changes, and possibly psychotic behavior characterize the behavioral challenges experienced by some individuals. Individuals suffering from HIV-associated dementia may develop these characteristics at various times and rates during the progression of the disease. HIV-associated dementia patients typically experience a high incidence of premature mortality due to or associated with their dementia. Dementia is a debilitating disease that literally steals the livelihood of its victims. Memory loss, depression, agitation, anxiety, and other adverse behaviors are caused by its apparently irreversible and destructive effects on the central nervous system.

These debilitating effects further reduce the life expectancy of HIV infected individuals. Working in concert, and without effective treatment, the virus and the dementia condition, destroy individual's immune systems, self-confidence, motor skills, and family relations. As a result, individuals with HIV-associated dementia experience premature mortality. In the absence of dementia, treatments for HIV affected individuals are given an opportunity to be more effective and possibly prolong the life of the individual. Further, in the absence of this condition the treatment may prove effective in retarding the replication of HIV and retarding its adverse effects. Increased intracellular calcium concentration in neurons and/or increased cortisol production by the adrenal, induced due to changes in hypothalamus-pituitary-adrenal (HPA) axis and probably glycoprotein 120 (gp 120) may trigger events that lead to HIV-associated dementia. (Corley PA. 1995; Corley PA. 1996; Simpson, David M; Brew, Bruce James 1999).

HIV, the virus whose progression leads to Acquired Immune Deficiency Syndrome (AIDS), is a retrovirus housed in a viral particle protected by various coat proteins, the most significant of which is glycoprotein 120 (gp|20). The gp|20 envelope facilitates infection of a host cell by binding to receptors on the surface of many immune cells such as T-cells as well as chemokine co receptors. After fusion of the viral particle with the host cell, replication of the viral particles is initiated and subsequent infection of other cells occurs. In addition to facilitating the introduction of HIV into host cells, research demonstrates that gp|20 is either directly or indirectly responsible for initiating HIV dementia. The direct hypothesis suggests that the gp|20 protein, which is often shed from the HIV virus after fusion occurs, interacts directly with chemokine receptors on the surface of neurons; thereby facilitating apoptosis and neuronal cell death. (Brew, Bruce James 1999). The indirect hypothesis suggests that apoptosis is caused by interaction of the HIV virus with non-neuronal cells of the central nervous system (CNS), specifically macrophages, microglia, and astrocytes. In this case, gp|20 facilitates the transport of HIV infected macrophages and microglia across the blood brain barrier (BBB), a selectively permeable membrane that prevents entry of foreign material. (Kaul, Marcus, et al. 2001). Once infected cells are in the brain, they release neurotoxins and promote a massive influx of calcium ion (Ca2+) into the neuron thus initiating apoptosis. (Smits, H. A. et al. 2000). HIV Infected macrophages, monocytes and microglia all release gp|20.

An abundance of gp|20 in the CNS disrupts the calcium homeostasis (Lipton SA, 1994) partly by reverting the glutamate uptake systems and by directly activating the NMDA subtype calcium channel-associated glutamatergic receptor and the calcium voltage-operated channels (Lipton SA, 1991). The induced massive calcium inward current leads to an impairment of the memory and learning processes and triggers the excitotoxicity cascade which leads to a neuronal death (Choi D W, 1992). Calcium ions facilitate intercellular communication through electrical polarization and depolarization and therefore opening a Ca2+ channel for too long is fatal to a neuron. (Epstein L and Gendelman H. May 1993).

A combination of both the direct and indirect interference of gp|20 with the calcium homeostasis may cause mitochondrial function impairment leading to critical cell death. (Simpson, David M.). At the same time, gp|20 indirectly induces an increase in blood and CSF cortisol concentrations leading to neurotoxicity and HIV-associated dementia. (Corley PA. 1995; Corley PA. 1996).

Chemokine receptors are also bound by the gp|20 envelope as co receptors with CD4 to permit entry into host cells. (Miller, Richard J. and Meucci, Olimpia 1999). This binding on cells of the CNS acts to stimulate and agonize the cells in an uncontrolled manner. Over stimulation subsequently acts to release glutamate and other neurotoxins and inflammatory cytokines resulting in neuronal death due to apoptosis. (Miller, Richard J. and Meucci, Olimpia. 1999).

Astrocytosis, proliferation of astrocytes, observed in patients with HIV, occurs when the virus retards the effectiveness of astrocytes to scavenge excess glutamate produced by infected macrophages and microglia. (Kaul, Marcus et al. 2001). Additional astrocytes are produced to compensate for the ineffectiveness of the cells. As a result of astrocytosis, more infected macrophages and microglia cross the BBB inducing massive neuronal death which leads to HIV-associated dementia. It is clear that the cause of HIV-associated dementia revolves around the activation of macrophages, microglia, chemokine receptors, and astrocytes within the CNS and subsequent apoptosis leading to dementia.

It is equally apparent that the process is made possible because the gp|20 envelope facilitates transfer of the HIV virus across the BBB and because cleaved gp|20 protein is able to interact with chemokine receptors on the surface of neurons. Prevalent theory also posits that HIV-associated elevation of cortisol levels is directly responsible for induction of HIV-associated dementia. The progression of HIV infection is accompanied by complex alterations in the production of adrenal steroids. HIV infection is associated with activation of the hypothalamic-pituitary-adrenal (HPA) axis function, leading to increased plasma and urinary cortisol levels. (Kumar M, et al. 2001; Kumar, M, et al. 2000).

Increased cortisol levels have been documented in both HIV-infected individuals and patients with AIDS. The increases in cortisol levels range from 20% to 50% above normal; the highest levels are found in patients with advanced disease. HIV-associated elevation of cortisol levels is hypothesized to have a major function in the pathophysiology of AIDS, including suppression of cell-mediated immunity and AIDS-associated dementia. (Corley PA. 1995).

In addition, there is experimental evidence suggesting that cortisol and its receptors are involved in the regulation of immune function in HIV infection. (Norbiato G, et al. 1997). Refaeli and colleagues have found a different line of evidence linking cortisol to AIDS. These investigators have shown that an HIV protein, the product of the vpr gene, mimics the actions of the glucocorticoids, including cortisol.

Previously, it had been shown that the vpr protein pierces the membranes of macrophages, the white cells that are among the first immune cells to host HIV infection. Refaeli's group has provided evidence suggesting that this HIV protein dupes the body into suppressing its own immune system. The vpr protein blocks the production of the Type 1 cytokines. In addition, the vpr protein was shown to induce healthy, uninfected T-lymphocytes in initiating programmed cell death. When these investigators added the steroidogenesis inhibitor, RU-486, to tissue culture cells, they found that it reversed the vpr protein's destructive effects on immunity. Furthermore, T-lymphocytes treated with the vpr protein and RU-486 continued to synthesize and secrete immune-boosting cytokines and did not succumb to programmed cell death.

Thus, HIV-associated elevation of cortisol levels maybe implicated in the pathophysiology of AIDS, including suppression of cell-mediated immunity and HIV-associated dementia as suggested by Corley. On the basis of this conclusion, Clerici and associates have postulated that preventing or reversing the cortisol: DHEA ratio and the induction of Type 1 cytokine production in patients with AIDS may serve to reduce programmed cell death and interfere with viral replication in HIV-infected cells. In contrast to the detrimental effects of high levels of cortisol in the pathologies described above, maintenance of the basal cortisol levels is necessary for the maintenance of basic biological functions.

Glucocorticoids regulate the metabolism of proteins, carbohydrates and lipids, and are essential to the adaptation to acute physical stressors (Munck et al, 1994). Development of compounds which block the excessive glucocorticoid synthesis without affecting the basal steroid formation has proven to be a difficult task because it requires the identification of a modulator of an activity rather than an inhibitor. Therefore, there is a need for additional treatments of a cortisol-mediated disease or disorder, including compositions for administration to a subject suffering from, or at risk of developing, a cortisol-mediated disease or disorder.

A faster onset of action, improved side-effect profile, reduced dosing amount and frequency, improved patient compliance, improved bioavailability and safety, and/or improved pharmacokinetic, pharmacodynamic, chemical and/or physical properties are also desired for the treatment of such conditions. Additionally, there is a need for methods and compositions that modulate levels of cortisol while maintaining basal cortisol levels. The discussion that follows discloses methods and compositions that help to fulfill these needs.

As more sophisticated hormone assay techniques for saliva were developed, more researchers became involved in the study of age and stress-related adrenal steroid circadian rhythm changes. Saliva provides a useful sample for cortisol/DHEA measurement in many cases because the level of steroids in saliva reflects that in blood. Recently, the analysis of salivary steroids is becoming a widely accepted screening tool for adrenal or gonadal function. Individual circadian rhythm has become more important than the absolute hormonal concentrations in disease diagnosis. Studies confirmed that salivary steroid levels reflect that of serum levels.

An extract of green coffee beans (commercially available under the trade name GCA®) contains a profile of at least 20 w/w % chlorogenic acids that have been shown to have a host of health benefits from LDL oxidation reduction, enhanced endothelial function aiding in hypertension reduction and general antioxidant function for reduced oxygen species activity in vivo. In addition, these specific polyphenols have been shown to have suppressive effects on the glucose 6-phosphatase pathway. This pathway is our body's primary pathway for the regulation and uptake of glucose into the cell walls. It is speculated or deduced that by controlling the regulation of glucose in this way we can assist the body with weight management, fatty acid synthesis activity and positively impact insulin activity.

While historical research supported the role these chlorogenic acids have on lowering the plasma glucose levels in response to oral dose administration, the science and clinical data was not clear on the specific mechanism of action associated with the secondary benefits seen in human subjects such as weight loss, increased energy, more satisfied disposition and mental health, increased sexual drive, etc. upon administration of the extract. Furthermore, synthetic derivatives of the chlorogenic acids did not carry any such secondary benefits in response to lower glycemic response.

Citation of the above documents is not intended as an admission that any of the foregoing is pertinent prior art. All statements as to the date or representation as to the contents of these documents is based on the information available to the applicants and does not constitute any admission as to the correctness of the dates or contents of these documents. Further, all documents referred to throughout this application are incorporated in their entirety by reference herein.

### SUMMARY OF THE INVENTION

The present disclosure provides methods for reducing cortisol levels in a subject human without the use of pharmaceutical preparations.

Specifically, it has been discovered that by reducing cortisol, GCA® administration can provide a source of enhanced adrenal and metabolic function, increase lean body mass by enhanced testosterone leading to increased muscle formation, increase libido, ovary function and elevated mood and brain receptivity.

In one embodiment, a method for reducing cortisol levels in a human subject is disclosed that includes the steps of generating a green coffee extract containing at least 20% chlorogenic acids by dry weight, generating a composition which includes the green coffee extract and is administrable to a human subject, and administering a therapeutically effective amount of the composition to the human subject, thereby reducing cortisol levels.

In other embodiments, disclosed is a composition for reducing cortisol level, said composition comprising a therapeutically effective amount of green coffee bean extract, said green coffee extract containing at least 20% chlorogenic acids by dry weight.

In further embodiments, disclosed is a method of treating a cortisol-mediated condition, disease or disorder, comprising administering to a subject human in need thereof an effective dose of green coffee bean extract, said extract containing at least 20% chlorogenic acids by dry weight.

One object of the present disclosure is to provide compositions for decreasing cortisol levels, which compositions comprise effective amounts of CGA®, said effective amounts ranging from approximately 1 mg to approximately 4000 mg per dosage. Another object is where such is administered to the human subjects at least twice a day for a period of at least two weeks.

Further, a method of treating a cortisol-mediated condition, disease or disorder comprising administering to a human subject in need thereof an effective amounts of CGA®, said effective amounts ranging from approximately 1 mg to approximately 4000 mg per dosage and administered to the human subjects at least twice a day for a period of at least two weeks has been presented.

Another object of the present disclosure is where the chlorogenic acids and related compounds include:
3-caffeylquinic acids (3-CQA) ranging from approximately 2% to approximately 15%;
5-caffeylquinic acids (5-CQA) ranging from approximately 5% to approximately 25%;
4-caffeylquinic acids (4-CQA) ranging from approximately 2% to approximately 15%;
5-feruloylquinic acids (5-FQA) ranging from approximately 1% to approximately 12%;
3,4-dicaffeylquinic acids (3,4-diCQA) ranging from approximately 1% to approximately 12%;
3,5-dicaffeylquinic acids (3,5 diCQA) ranging from approximately 1% to approximately 12%; and
4,5-dicaffeylquinic acids (4,5 diCQA) ranging from approximately 1% to approximately 12%.

According to still further features in the described preferred embodiments, the composition described herein is in the form of a supplement, beverage, food, tea, a tincture, a concoction, an infusion tablet, a capsule, a pill, a bar, a chewable gum, a dissolvable oral strips, a lotion, a powder or granules.

According to still further features in the described preferred embodiments, there is provided a dietary and/or pharmaceutical composition comprising an herbal composition described herein and a dietetically and/or pharmaceutically acceptable excipient.

It has been found that the greatest increase in saliva cortisol levels occurred in patients treated with the dosage of GCA® 350 mg daily for a period of approximately two (2) weeks. Administered orally GCA has been shown to decrease cortisol levels by as much as 47% in human subjects. Subsequently, this elevation in DHEA levels has been associated with increased energy and metabolism, weight loss, increased subject testosterone levels and lean muscle mass, feelings of happiness, satisfaction and sexual desire as well as elevated serotonin levels. In some embodiments, cortisol levels may be reduced and conditions associated with mood disorders or neurotransmitter functions may be treated or alleviated.

According to one embodiment of the present disclosure, GCA® can also be applied topically as a serum, cream, lotion, oil, gel or patch. Applied topically, GCA® has been shown to enhance cell turnover and decrease the physical signs of skin aging, such as skin UV damage, wrinkles, lines, and scarring.

### BRIEF DESCRIPTION OF THE DRAWINGS

The following figures are included to illustrate certain aspects of the present invention, and should not be viewed as an exclusive embodiments. The subject matter disclosed is capable of considerable modification, alteration, and equivalents in form and function, as will occur to one having ordinary skill in the art and the benefit of this disclosure.

FIG. 1 is a chart of a Visia® analysis relative to control group for each skin parameter measured, according to one or more embodiments.

FIG. 2 depicts statistics for a control group and a coffee extract group, according to one or more embodiments.

FIG. 3 depicts product performance for various skin parameters, according to one or more embodiments.

### DETAILED DESCRIPTION

### Examples

The following examples are illustrative of the present disclosure and parts and percentages are by dry weight unless otherwise indicated. It should be noted that these examples are only that—examples—a wide range of conditions, which together with the above descriptions, illustrate the invention in a non limiting fashion.

### Example 1

Twelve human subjects, 6 female and 6 male subjects were recruited to evaluate the affect of oral administration of GCA® on recirculating hormones. The twelve subjects were initially screened for any preexisting conditions, potential drug interactions, health issues and current supplement and or drug usage. All subjects were in good health and currently not taking any sort of treatments that were known to have an impact on the tested hormones. Each subject was requested to provide saliva samples for an initial hormone's testing to determine baseline hormone levels. A complete hormone panel test which includes Estrogen, Testosterone, DHEA, and am, mid-pm and pm Cortisol levels were completed by Labrix Clinical Services. After baseline readings were completed GCA® having the following chlorogenic acid composition was orally administered at 350 mg, twice daily for four weeks:

### 3-CQA=6.78%

### 5-CQA=20.9%

### 4-CQA=8.31%

### 5-FQA=3.62%

3,4-diCQA=3.65%
3,5-diCQA=4.56%
4,5-diCQA=2.88%

After four weeks, a second set of saliva samples were submitted by each subject for additional hormone testing on the same parameters measured during the baseline readings. The baseline and subsequent results after the 4 weeks of GCA administration are shown in Tables 1 and 2.

Five of the six female subjects also completed a quality of life questionnaire to determine if they observed any changes in their physical and or emotional state during the trial period. The scores were ranked 0-10 with 0 being the lowest state and 10 being the highest. The female subjects were asked to rank their energy levels, as determined by their ability to maintain energy throughout the day, satisfaction in their overall weight, energy and mood, sex drive by their number and days they initiate sexual intimacy with their life partner, motivation by their overall willingness to take on new projects, try new things or aide others in performing a task and happiness by their overall feeling of being in a happy state of mind throughout the day. The quality of life scores for all five subjects is provided in Table 3.

HORMONE PANEL RESULTS (mean and percent change among subjects) (Table 1):

TABLE 1

| Hormone | Baseline | Week 4 | % Change |
|---|---|---|---|
| Female DHEA | 162.46 | 763.47 | 370 |
| Female Testosterone | 31.73 | 55.12 | 74 |
| Male DHEA | 179.45 | 463.45 | 158 |
| Male Testosterone | 61.78 | 76.3 | 23 |

CORTISOL Results (mean and percent change among all subjects) (Table 2):

TABLE 2

| Hormone | Baseline | Week 4 | % Change |
|---|---|---|---|
| Female AM | 17.19 | 9.17 | −47 |
| Cortisol | | | |
| Female PM | 5.204 | 2.81 | −46 |
| Cortisol | | | |
| Male AM | 13.19 | 14.41 | 9 |
| Cortisol | | | |
| Male PM | 1.54 | 1.78 | 16 |
| Cortisol | | | |

Quality of Life Scores (rated from 0-10, reported as mean percent change in all subjects studied (Table 3)

TABLE 3

| QOL Parameter | % Change |
|---|---|
| Energy | 159 |
| Satisfaction | 109 |
| Sex Drive | 64 |
| Motivation | 99 |
| Happiness | 102 |

The average increase in recirculating DHEA levels was 370%; testosterone increases an average of 74% and cortisol levels declined by 47% in the female subject group. In the male group the results were not as profound but just as promising. The DHEA levels increased by an average of 158%, testosterone by 23% while cortisol levels increased slightly.

This increase in DHEA levels and decrease in cortisol levels also led to a corresponding increase in energy levels by 159% as reported by all female subjects, as well as an increase in motivation 99%, satisfaction 109%, happiness 102%, and sex drive by 64% in all of the female subjects who completed the Quality of Life questionnaire.

The benefits in quality of life scores, such as motivation, mood, and happiness can further be supported by evidence that cortisol level reduction and regulation has been associated with supplemental cognitive benefits. Research by Stranahan et al out of John Hopkins Department of Psychological and Brain Sciences demonstrated that lowering corticosterone levels reversed deficits in brain-derived neurotropic factor (BDNF) and TrkB expression in the hippocampus. A number of neuronally relevant hormones such as insulin and cortiocosterone can normalize fasting glucose levels by regulating adrenal function. This cause and effect is similar to what has been achieved in human subjects upon oral administration of GCA, green coffee extract. For example, utilizing GCA as the active for regulation of glucose also demonstrates lowering and normalizing of recirculating cortisol levels and therefore we might deduce can also have a similar impact on BDNF. Research suggests that this positive effect on BDNF after ingestion of coffee compounds was elucidated by the procyanidins, and not the polyphenolic acids. Therefore these results are both surprising and also lead to a suggestion that mood, energy and cognitive health may have linked stimulation by the GCA, green coffee extracts, rich in a unique profile of chlorogenic acids, effectiveness on maintaining healthy adrenal function and glucoregulation pathways.

In one study, GCA administration has the effect of positively down regulating insulin activity. Individuals that were tested had a predisposition for diabetes, such as elevated BMI or other biological markers for diabetes. It one test, administration of 350 mg of GCA three times per day for twelve weeks showed these results of lowering or reducing insulin activity.

### Example 2

Forty two female subjects with a Fitzpatrick skin type between I-III were randomly assigned into four different participant groups. The four groups were requested to apply one of the following topical regimen protocols daily for 12 weeks:

1. Group 1 (Brown)—control group applying a Neutrogena Moisturizer 2λ daily and Tretinoin Cream 0.025% applied 1× daily.
2. Group 2 (Green)—group applying a Green coffee extract, CGA® 2× daily.

Both groups were also asked to cleanse their face 2× daily prior to application of the serums, in addition the am routine included applying sunscreen spf 30 or greater. All subjects visited the Raval Aesthetician clinic every four weeks. Throughout the course of the clinical and upon each visit each subject was given a skin analysis with Visia® technology, requested to complete a performance questionnaire and photographs were taken to monitor skin progress in visual appearance.

The Visia® analysis reported the following parameters: change in facial spots, UV damage, wrinkles, texture, pores and porphiryns. Data provided a snapshot of the individuals skin performance relative to other subjects within the database of the same skin type as well as scoring assessment for each parameter to measure against that subjects baseline at time t=0.

The performance questionnaire contained questions each subject experience rating the following parameters on a scale 1-5: overall appearance, skin tone, skin clarity, skin texture, moisture and skin elasticity.

All data with the exception of the level of porphiryns was analyzed relative to the control and corrected for ANNOVA to standard for an individual's baseline as well as significant changes with group outcome. Porphiryns were excluded in most analysis relative to control due to issues with product handling and lack of preservatives within the product which promoted elevated bacterial counts that would presumably not be present had topical contained additional ingredients to control bacteria and product contamination upon handling.

Visia® analysis (Table 4) demonstrated an improvement of at least three skin parameters measured for all subjects: spots, UV damage and pores. Addressing each parameter an improvement in skin spots was most prominent within the green coffee extract (GCA®) group by 8%. UV damage which was a critical measurement in performance outcome demonstrated the most promising benefits in both the green coffee extract, GCA® (5%).

TABLE 4
Visia ® Analysis. Data reported

| | Control | GCA |
|---|---|---|
| Spot Reduction | 4% | 6% |
| UV Damage Reduction | 1% | 5% |
| Wrinkle Reduction | −10% | 22% |
| Texture Improvement | −1% | 12% |
| Pore Reduction | 7% | 10% |

on average percent change relative to individual's baseline, (p<0.005)

Relative to the control, the GCA® topical had statistically significant benefits in skin performance outcome (FIG. 1) for UV damage, wrinkles and skin texture. There was a strong and positive correlation between the change in UV damage and the amount of wrinkles and overall skin texture as measured by Visia® leading to the link between antioxidant benefit/performance and long term skin health and visual repair.

FIG. 1 is a chart 100 of a Visia® analysis relative to control group for each skin parameter measured. Data reported on average percent increase in skin parameter measured relative to the control group corrected for individuals baseline values (p<0.005).

Important to long term compliance and ultimately product performance is the perception of skin improvements with the subjects. Therefore a skin performance questionnaire become an integrate part of the review association with skin performance and perception of value to the subject. As was seen within the Visia® analysis the survey results also are indicative of a statistically significant improvement in all skin parameters assessed by each individual. The group using the Green Coffee Extract objectively scored an overall 31% improvement relative to their baseline reporting, with significant improvements in skin clarity and elasticity.

TABLE 5
Product Quality Questionnaire as assessed by the individual. Data
reported on average percent increase in performance of skin parameter,
(p < .002) against individuals baseline data value.

| | Control | Coffee Extract |
|---|---|---|
| | (21%) | (31%) |
| Appearance | 22% | 35% |
| Texture | 20% | 27% |
| Skin Tone | 25% | 19% |
| Skin Clarity | 15% | 36% |
| Moisture | 20% | 29% |
| Elasticity | 25% | 42% |

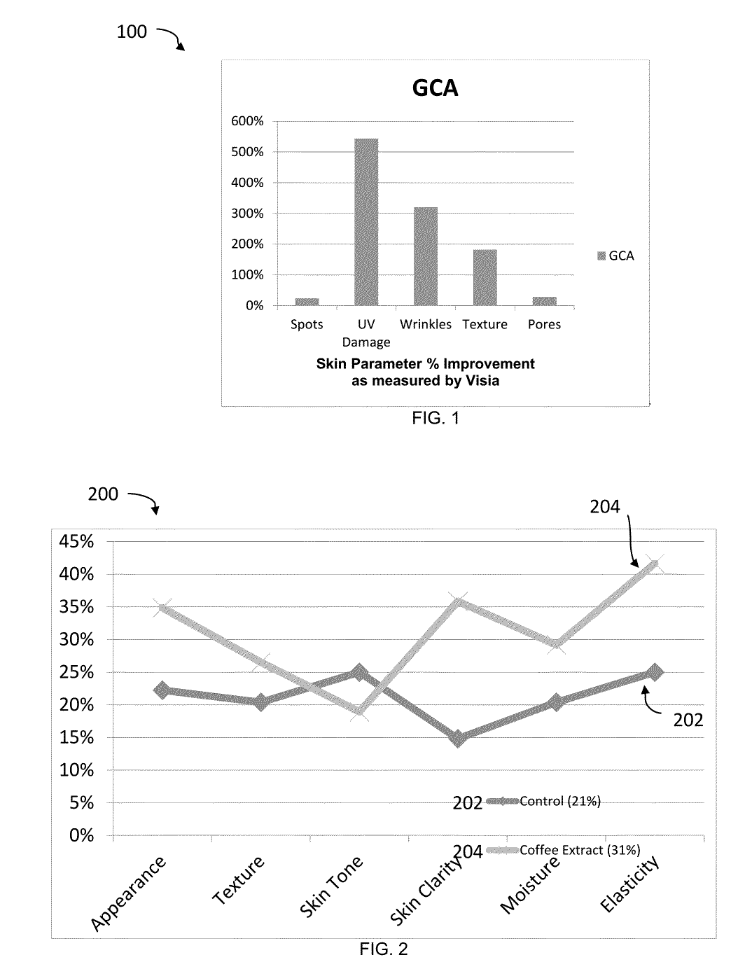

Chart 200 of FIG. 2 depicts statistics resulting from a produce quality questionnaire as assessed by the individual for a control group 202 and a coffee extract group 204. Chart 200 indicates a visual perspective of product performance for each skin parameter for each product applied. Overall all subjects also reported an increase in product performance relative to both their baseline data and the control group, with the order of improvement of each topical being Control, Glucarate, Combination, Green Coffee Extract groups.

Data reported on average percent increase in performance of skin parameter, (p<0.002) against individuals baseline data value.

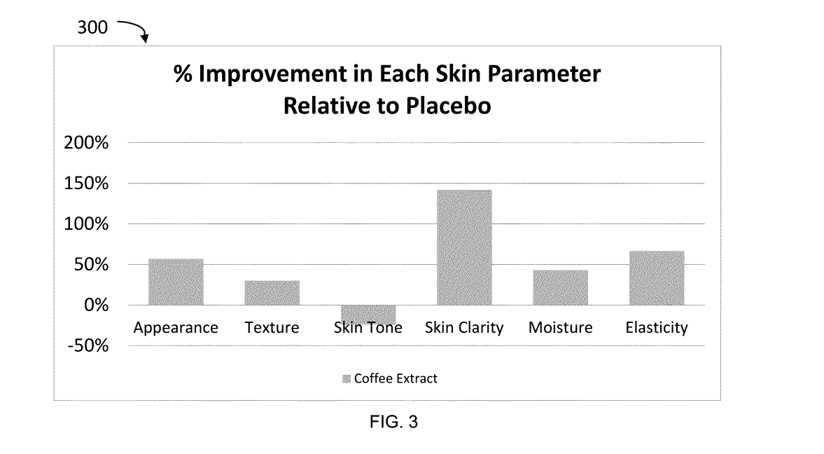

Relative to the control group there were varying changes in the product performance described by the individual. Chart 300 of FIG. 3 is a subject questionnaire results reporting product performance for each skin parameter. Data is expressed as average value relative to control for each skin parameter (p<0.002).

It has been reported within the literature that topical application of a low dose retinoic acid applied daily can have positive benefits to skin performance through enhanced skin cell turnover and ultimately lead to skin repair. Specifically, as it relates to the negative effects of photo damage retinoic acid has been shown when applied topically, to decrease UV damage, increase skin collagen, and reduce wrinkles. We have been able to demonstrate that applying additional topical antioxidants in the form of green coffee beans, GCA® can have an enhanced effect on this current dermatology treatment. In addition to reducing the effects of photo damage, the green coffee extract showed significant improvements in skin clarity and added moisture, resulting in enhance skin elasticity.

In conclusion, green coffee bean extract, GCA® has been shown to positively impact skin performance, skin aging and the visual impact on skin health. Specifically, clinical review demonstrated that topical application of antioxidants in the form of green coffee extracts standardized to 50% chlorogenic acids provided a 32% increase in healthy skin parameters as measured by Visia® analysis. The most significant benefit came with applying the green coffee extract resulting in reduction in UV damage (543%) and wrinkles (320%) in comparison with a topical containing Tretinoin Cream 0.05%. GCA®, green coffee extract at 6 w/w % as a topical agent, was well tolerated. No adverse events and skin conditions were reported.

### Example 3

An antioxidative power (AP) method offers the determination of the all over antioxidative power of active ingredients, i.e. plant extracts, etc., by following the reducing activity against a stable test radical—diphenyl-picryl-hydrazyl (DPPH) with ESR spectroscopy. Jung K, Richter J, Kabrodt K, Lucke I M, Schellenberg I, Herrling T. The antioxidative power AP—A new quantitative time dependent (2D) parameter for the determination of the antioxidant capacity and reactivity of different plants. Spectrochim Acta A Mol Biomol Spectrosc. 63(2006):846-50; Jung K, Sacher M, Blume G, Janβen F, Herrling T. How Active are Biocosmetic Ingredients? SÖF W—Journal 133 1/2—2007. The AP method basically utilizes the well known DPPH method with a major difference that both the antioxidative capacity and the antioxidative activity are used to characterize the antioxidant under discussion. For this purpose different concentrations of the active ingredient, GCA along are investigated by ESR spectroscopy and the decrease of the test radical spins is tracked accordingly for each set. With this innovative technique important kinetic information is additionally obtained which is completely neglected by most of the other tests systems. The method is robust and qualitative because both the reaction time and the reduction potential of the antioxidants contribute to the calculation of the AP, where AP=no free radicals/mg*min.

The resulting AP is expressed in antioxidative units (AU), where 1 AU corresponds to the activity of a 1 ppm solution of pure vitamin C (ascorbic acid) as a benchmark. This method allows a rapid and general applicable technique for the measurement of the AP of very different classes of substances

The measurements discussed in this example were performed with the X-band ESR spectrometer Miniscope MS 300 (Magnettech, Germany) and the following technical parameters: 60 G sweep width, 100 Gain, 1 G modulation amplitude, 7 mW attenuation, 3365 G central field, 0.14 sec time constant. At least 3 concentrations of the test sample were prepared and added to DPPH to obtain an initial radical concentration of 0.1 mM.

The Antioxidant Power determines the activity of antioxidants and radical scavengers. The higher the capacity of a test substance to neutralize free radicals is and the higher the reaction velocity is, the higher is the AP. The AP values are benchmarked against ascorbic acid (vitamin C) and expressed in Antioxidative Units (AU). The values for the GCA samples and those already published within the literature are outlined below in Table 6.

The GCA Green Coffee Extract showed a very high antioxidative capacity and free level reactivity. Compared to other green coffee extracts as seen in Table 6 the concentration of the GCA actives is elevated. This is a novel and unique finding which demonstrates that some synergistic activates among the compounds has the ability to allow for high ROS activity thus demonstrating its anti-inflammatory properties and further supporting the decreased effects of declined cortisol production at the cellular level. Since glucocorticoids such as cortisol have been shown to decrease cytochrome c oxidase activity and to induce apoptosis in various cell lines one could speculate that the enhanced AP because the polyphenols are free to induce ROS activity as oppose to mitigating the effects typically experienced due to cellular cortisol activity.

TABLE 6
Antioxidant Power of Commercial Grade Green Coffee Extracts

| Product ID | AP | t ® (min) |
|---|---|---|
| GCA green coffee extract | 239.785 | 0.22 |
| Green coffee extract Ref 1 (liquid) | 5.069 | 0.26 |
| Green coffee extract Ref 2 (liquid) | 11.747 | 0.77 |
| Green coffee extract Ref 4 (solid) | 72.755 | 0.16 |

Although the invention has been described with reference to specific embodiments, this description is not meant to be construed in a limited sense. Various modifications of the disclosed embodiments, as well as alternative embodiments of the invention will become apparent to persons skilled in the art upon the reference to the description of the invention. It is, therefore, contemplated that the appended claims will cover modifications that fall within the scope of the invention.

What is claimed is:

1. A method for reducing cortisol levels in a human subject, said method comprising:
generating a green coffee extract containing at least 20% chlorogenic acids by dry weight;
generating a composition which includes said green coffee extract and is administrable to a human subject; and
administering a therapeutically effective amount of said composition to said human subject, thereby reducing cortisol levels.

2. The method of claim 1 wherein a dosage of said green coffee extract is provided in a range from approximately 1 mg to approximately 4000 mg per dosage.

3. The method of claim 1 wherein an optimum dosage unit is provided in a range from approximately 250 mg to approximately 400 mg per dosage.

4. The method of claim 1 wherein said composition is administered orally.

5. The method of claim 1 wherein said composition is administered topically.

6. The method of claim 1 wherein said chlorogenic acids and a related compounds include:
3-CQA=2-15%
5-CQA=5%-25%
4-CQA=2%-15%
5-FQA=1%-12%
3,4-diCQA=1%-12%
3,5-diCQA=1%-12%
4,5-diCQA=1%-12%

7. The method of claim 1 further comprising treating a condition of said human subject, said condition being selected from the group comprising a low energy level, a low libido, an increased body mass index, and an increased percent body fat.

8. The method of claim 1 wherein said reduction of cortisol level ameliorates symptoms related to at least one of a decreased energy level, decreased libido, decreased motivation, decreased overall happiness, and decreased life satisfaction.

9. The method of claim 1 wherein said reduction of cortisol level decreases at least one of a body mass index and a percent body fat among said human subjects.

10. The method of claim 1, wherein said therapeutically effective dose is administered daily in said human subject for a period of at least two weeks.

11. A composition for reducing cortisol level, said composition comprising a therapeutically effective amount of green coffee bean extract, said green coffee extract containing at least 20% chlorogenic acids by dry weight.

12. A method of treating a cortisol-mediated condition, disease or disorder, comprising administering to a subject human in need thereof an effective dose of green coffee bean extract, said extract containing at least 20% chlorogenic acids by dry weight.

13. The method of claim 1, further comprising treating a disease or condition associated with at least one of adrenal fatigue and insulin resistance.

14. The method of claim 13 wherein a dosage of the green coffee extract is provided in a range from approximately 1 mg to approximately 4000 mg per dosage.

15. The method of claim 13 wherein an optimum dosage unit is provided in a range from approximately 250 mg to approximately 400 mg per dosage.

16. The method of claim 13 wherein the composition is administered orally.

17. The method of claim 1, wherein the reduction of cortisol improves at least one of said person's memory, cognition, and mood.

18. The method of claim 1, wherein said reduction of cortisol levels enhances adrenal function, wherein said enhanced adrenal function improves glucose regulation, thereby reducing insulin activity.

19. The method of claim 1, wherein said reduction of cortisol levels increases serum levels of brain-derived neurotropic factor (BDNF).

20. The method of claim 1, wherein said reduction of cortisol levels increases serum levels of brain-derived neurotropic factor (BDNF), thereby improving at least one of said persons memory, cognition, and mood.
